(19) United States
(12) Patent Application Publication
(10) Pub. No.: US 2013/0211415 A1
(43) Pub. Date: Aug. 15, 2013

(54) STEERABLE TISSUE MANIPULATION MEDICAL DEVICES AND RELATED METHODS OF USE

(71) Applicants: Jeffrey Zerfas, Bloomington, IN (US); Jennifer Keeler, Hopkinton, MA (US)

(72) Inventors: Jeffrey Zerfas, Bloomington, IN (US); Jennifer Keeler, Hopkinton, MA (US)

(73) Assignee: BOSTON SCIENTIFIC SCIMED, INC., Maple Grove, MN (US)

(21) Appl. No.: 13/762,736

(22) Filed: Feb. 8, 2013

Related U.S. Application Data
(60) Provisional application No. 61/596,765, filed on Feb. 9, 2012.

(51) Int. Cl. A61B 17/00

(52) U.S. Cl. CPC A61B 17/00234
USPC 606/114; 606/1; 606/198; 606/205

(57) ABSTRACT

A medical device and related methods of use are disclosed. The medical device includes a tubular member having a proximal end, a distal end, and a lumen extending between the proximal end and the distal end. The medical device further includes an elongate member having a proximal end and a distal end, such that at least a portion of the elongate member is movably disposed within the lumen of the tubular member. The distal end of the elongate member includes an end-effector, wherein a portion of the elongate member between the proximal end and the distal end transitions automatically from a first state to a second state when the portion is advanced out of the lumen of the tubular member, and wherein the portion is proximal of the end-effector.

### CROSS-REFERENCE TO RELATED APPLICATIONS

This application claims the benefit of priority of U.S. Provisional Application No. 61/596,765, filed Feb. 9, 2012, which is incorporated by reference herein in its entirety.

### FIELD

Embodiments of the present disclosure relate generally to medical devices suitable for use in surgical procedures. In particular, embodiments of the instant disclosure relate to minimally invasive steerable medical devices employed for, among other things, body tissue manipulation and/or retrieval procedures.

### BACKGROUND

Minimally invasive medical procedures typically involve introducing certain instruments, such as endoscopes or catheters, into a patient's body through natural body openings or small percutaneous incisions. These instruments may then be urged distally through cavities or lumens in the body to a desired site. Once at a desired location, various medical devices, such as suction pumps, cauterization tools, graspers, clippers, lasers, baskets, lithotripters, forceps, biopsy devices, or snares may be inserted through the endoscope or catheter, allowing operators to perform procedures within the patient's body without causing massive trauma.

Most of these procedures are directed toward unwanted stone or tissue removal. For example, in a lithotripsy procedure, acoustic waves are applied to rupture a stone into smaller fragments. Then surgeons typically insert a retraction device for removing the fragments. Sometimes fragments may lodge in capillaries or crevices of body lumens, and an endoscope, though flexible, may not allow the surgeon to easily reach a fragment, leading to excessive procedure time. If the operator cannot reach the exact location, the fragment may remain in the patient's body, increasing the likelihood of infection. The alternatives, such as relocating devices for retrieving fragments, increase procedure time and cost.

Thus, there exists a need for devices with flexible distal ends allowing easier approach to target areas inside body lumens, which allow operators to conduct safer, quicker, and more efficient medical procedures.

### SUMMARY

Embodiments of the disclosure provide a medical device with an articulable (e.g., steerable) end-effector.

In accordance with an aspect of the present disclosure, a medical device may include a tubular member having a proximal end, a distal end, and a lumen extending between the proximal end and the distal end. The medical device may further include an elongate member having a proximal end and a distal end, wherein at least a portion of the elongate member is movably disposed within the lumen of the tubular member. The distal end of the elongate member may include an end-effector, wherein a portion of the elongate member between the proximal end and the distal end is configured to transition from a first state to a second state when the portion is advanced out of the lumen of the tubular member, and wherein the portion is proximal of the end-effector.

In various embodiments, the medical device may include one or more of the following features: the portion may include a shape memory or super-elastic material; the end-effector may be selectively steerable; the portion may be configured to transition from the first state to second state without the application of any additional forces; the first state may be substantially straight; the second state may include a curved configuration; the portion may be configured to transition from the first state to the second state upon exposure to a trigger; the trigger may be one of body chemistry or temperature; the end-effector may be configured to manipulate tissue or other objects within a patient's body; and the end-effector may include a self-expanding basket.

In accordance with another aspect of the invention, a medical device may include a tubular member having a proximal end, a distal end, and a lumen extending between the proximal end and the distal end. The device may further include an elongate member including a shape-memory material and having a proximal end and a distal end, wherein at least a portion of the elongate member is movably disposed within the lumen of the tubular member, wherein the distal end of the elongate member includes an end-effector, wherein a portion of the elongate member between the proximal end and the distal end is configured to transition from a substantially straight configuration to a second configuration different from the substantially straight configuration when the portion is advanced out of the lumen of the tubular member, and wherein the portion is proximal of the end-effector.

In various embodiments, the medical device may include one or more of the following features: the second configuration may include the distal end of the elongate member deflecting away from a longitudinal axis of the medical device; the portion may be configured to transition from the substantially straight configuration to the second configuration upon exposure to a trigger; the trigger may be one of body chemistry or temperature; the end-effector may be expandable; the elongate member may further includes a lumen; and the end-effector may be selectively steerable.

In accordance with an alternate aspect of the disclosure, a method of manipulating tissue may include introducing a medical device into the body. The medical device may include a tubular member having a proximal end, a distal end, and a lumen extending between the proximal end and the distal end. The medical device may further include an elongate member having a proximal end and a distal end, wherein at least a portion of the elongate member is movably disposed within the lumen of the tubular member. The distal end of the elongate member may include an end-effector, wherein a portion of the elongate member between the proximal end and the distal end is configured to transition from a first state to a second state when the portion is advanced out of the lumen of the tubular member, and wherein the portion is proximal of the end-effector. The method may further include advancing the medical device to a desired location within the body, deploying the elongate member by sliding the elongate member relative to the tubular member such that the portion transitions from the first state to a second state, and manipulating the end-effector.

In various embodiments, the portion is substantially straight in the first state and substantially curved in the second state; and the method may further include grasping tissue with the end-effector.

### BRIEF DESCRIPTION OF THE DRAWINGS

The accompanying drawings, which are incorporated in and constitute a part of this specification, illustrate embodiments of the present disclosure and together with the description, serve to explain the principles of the disclosure.

FIG. 1A is a partially sectioned side view of an exemplary integrated medical device, according to embodiments of the present disclosure.

FIG. 1B is a partially sectioned side view of an exemplary distal end of the device of FIG. 1A in a first state.

FIG. 1C is a partially sectioned side view of an exemplary distal end of the device of FIG. 1A in the process of extending toward the a second state.

FIG. 1D is a partially sectioned side view of an exemplary distal end of the device of FIG. 1A in the second state.

FIG. 1E is a partially sectioned side view of an exemplary distal end of the device of FIG. 1A in an extended but compressed configuration. Him

FIG. 2 is a partially sectioned side view of another embodiment of an end-effector, according to the present disclosure.

FIGS. 3A and 3B are partially sectioned side views of further embodiments of end-effectors, according to the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts.

### Overview

Embodiments of the present disclosure are directed to steerable medical or otherwise selectively positionable devices for endoscopes or catheters. In one embodiment, the device includes a semi-rigid, rigid, or flexible elongate member, which may comprise an elongate tube, provided with a pre-formed distal bend, such as, for example, a 45 degree bend in a bend portion of the elongate member. An axially slidable sleeve is slidably received over the elongate member to allow the bent portion to be straightened by retracting the elongate member into the sleeve.

In accordance with the present disclosure, the medical device may be inserted into the body by first inserting the device into a channel of an endoscope or catheter, or by inserting the device directly into a body lumen or incision.

The elongate member, in one embodiment, may include a Nitinol™ tube having a preformed 45 degree bend proximal to the distal end with an end-effector provided at its distal end. In other embodiments, the elongate member may be made of any material capable of returning to a pre-formed configuration. Exemplary end-effectors may include, but are not limited to, an expandable basket or cage, a mesh, graspers, scissors, a snare loop, forceps, lithotripter, or pincers. The end-effector may be secured to the distal end of the elongate member, or if the elongate member is implemented as a hollow inner tube defining a lumen, medical tools may be passed through the lumen of the elongate member to extend from the distal end, thereby defining end-effectors at the distal end of the elongate member.

The end-effector may be used in a dissection procedure, such as prostate tissue removal, bladder stone removal, or PCNL (Percutaneous Nephrolithotomy). The end-effector may thus be implemented as a resection or dissection tool or a device for retracting tissue dissected by a dissection tool.

### Exemplary Embodiments

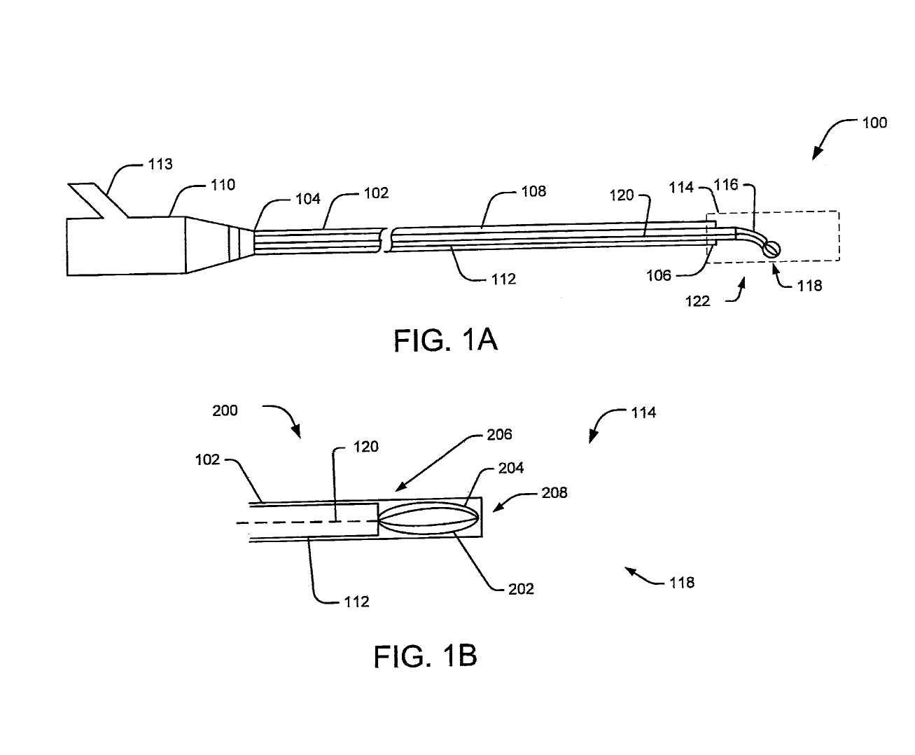

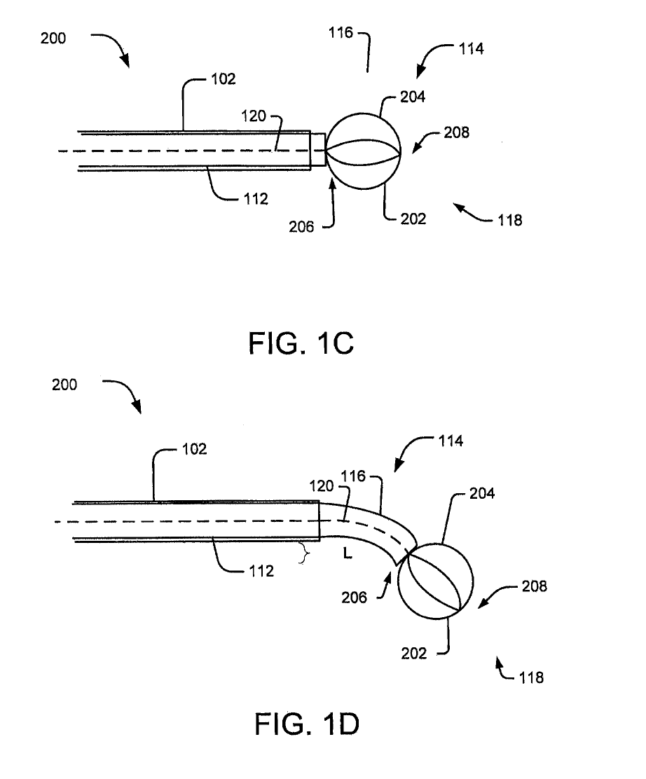

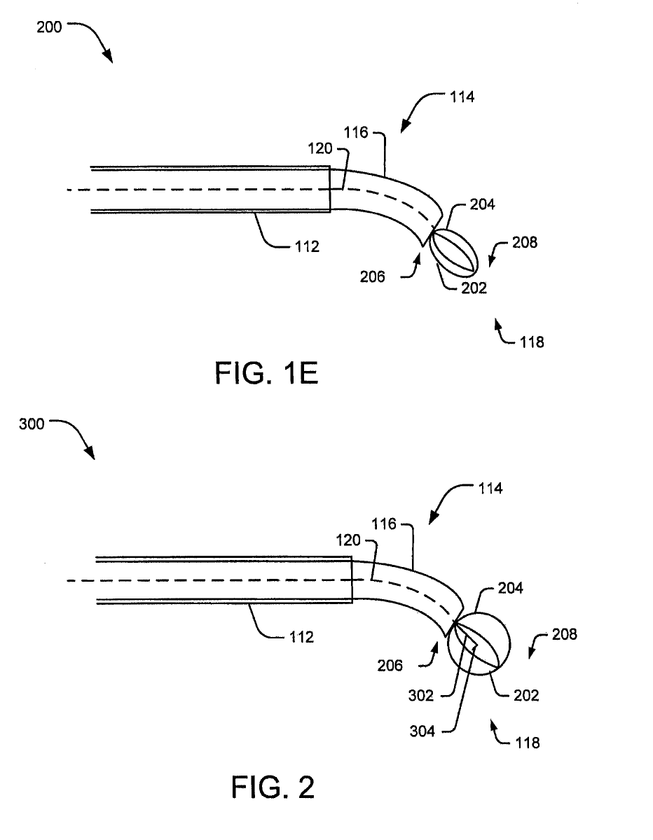

FIG. 1A illustrates an exemplary medical device 100 according to one embodiment of the present disclosure. The device includes an elongate outer tube or sheath 102 having a proximal end 104, a distal end 106, and a lumen 108 formed between the proximal and distal ends. The medical device 100 in this embodiment further includes a handle 110 coupled to the sheath's proximal end 104 and an elongate member 112 slidably received in the sheath 102 and having an end-effector 114 at the medical device's distal end 122. The elongate member 112 includes a preformed bend at a portion 116 proximate to the end-effector 114.

The sheath 102 may include a substantially circular cross-section, and may be made of a suitable biocompatible material, such as stainless steel, polyurethane, plastic, or other suitable materials to provide the features defined below. Other suitable cross-sectional shapes and methods of manufacture could be adopted as is known to those skilled in the art. The sheath 102 may be flexible along a portion of its length or it may be semi-rigid, or, sheath 102 may be rigid along its entire length, or along a portion of its length. Flexibility allows sheath 102 to maneuver circuitous turns in the patient's body, while rigidity allows the sheath 102 to exert the necessary force, for example, to urge the medical device 100 forward and/or retain elongate member 112 in a desired configuration, as is described below in greater detail. Insofar as the inner tube or elongate member 112 is provided with a pre-formed distal end portion, at least the distal end portion of the sheath 102 must possess sufficient rigidity to force elongate member 112 into a straightened configuration when the member is retracted into the outer sheath 102. In some embodiments, elongate member 112 and/or distal end 106 may include articulable segments secured to one another. Steering means, such as mechanical or electrical actuators may be present on the handle 110 to steer the sheath 102. Sheath steering means are widely known in the art, and any of these means may be utilized without departing from the scope of the present disclosure.

A lubricious coating may be applied to the outer surface of sheath 102 to facilitate insertion into a body lumen or an endoscopic device. In some embodiments, the lubricious coating may be applied to the inner surface of sheath 102 to facilitate sliding of elongate member 112. Further, in order to detect the position of the medical device 100 within a patient's body, at least some portions of the sheath 102, elongate member 112, and/or end-effector 114 may include radiopaque materials, such as, e.g., gold, palladium, platinum, tantalum, tungsten alloy, or polymeric materials loaded with radiopaque agents such as barium sulfate (BaSO₄) or bismuth subcarbonate ((BiO)₂CO₃). Radiopaque materials are understood to be materials capable of producing a relatively bright image on a fluoroscopic monitor or other imaging device.

The sheath 102, elongate member 112, and end-effector 114 may further be coated with an antibacterial coating to inhibit bacterial growth on their surfaces. The antibacterial coating may contain an inorganic antibiotic agent, disposed in a polymeric matrix that adheres the antibiotic agent to the sheath 102, end-effector 114, or any suitable surface of medical device 100. The lumen 108 may include one or more working channels (not shown) extending from the proximal end 104 to the distal end 106. Operators may insert different medical tools into the working channels. For example, an operator may place a cutting tool in one channel, and a retraction tool in another. In some embodiments, imaging and/or illumination devices such as fiberoscope, or energy devices such as laser fibers, lithotripters may be inserted through channels. It is understood that other exemplary tools may be inserted in the working channels without departing from the scope of the present disclosure.

The elongate member 112 may have a substantially circular cross-section, and it may be made of any suitable material including, but not limited to, shape memory or superelastic materials. Such materials include, e.g., Nitinol™ or Elgiloy™. Other suitable cross-sectional shapes, such as elliptical, oval, polygonal, or irregular, are also contemplated, as may be useful in particular scenarios. The elongate member 112 may be flexible along a portion of its length.

FIGS. 1B-1D are partial cutaway detail views of the distal end of medical device 100, within the box shown in FIG. 1A. These drawings illustrate the first and second states, respectively, of the elongate member 112. In general, the first state, shown in FIG. 1B, has both elongate member 112 and end-effector 114 entirely within sheath 102. Here, and continuing with the remainder of the discussion in connection with FIGS. 1B-1E, end-effector 114 may be a basket 202, discussed in greater detail below. Basket 202 may be laterally compressed into a collapsed configuration, so that its diameter fits within sheath 102, and its distal tip is proximal of the distal tip of sheath 102. FIG. 1C shows elongate member 112 extending distally relative to sheath 102. As shown, the distal end of elongate member 112 has cleared the distal end of sheath 102, and basket 202 has expanded to its full diameter. FIG. 1D illustrates the second state, in which elongate member 112 has extended to the point where bend portion 116 is extended past the distal end of sheath 102, allowing elongate member 112 to bend accordingly. Essentially, as elongate element 112 is advanced further from the distal end of sheath 102, more of the bend portion 116 is free from the constraints of sheath 102, and accordingly the distal portion of the elongate element 112 assumes an increasing angle, in accordance with a preformed configuration of elongate element 112. As a result, the elongate element's distal tip 206 moves laterally away from the longitudinal axis of the medical device 100, achieving a maximum lateral distance L when bend portion 116 is fully clear of sheath 102. This general description will be amplified below.

Transition from the first state to the second state of elongate element 112 occurs due to the action of the preformed bend portion 116. A bend is applied to the distal portion of elongate member 112 during manufacture. Suitable manufacturing techniques to provide the requisite bend are well known in the art. In some embodiments, a superelastic or shape memory material can be chosen, capable of bending between the relatively straight position of FIG. 1B to the fully angled position of FIG. 1D without plastic deformation. In another embodiment, a shape memory material, such as Nitinol™ or Elgiloy™ can be employed. That embodiment requires formation of the angle as the basic shape of the material. Insertion of the resulting elongate member into the sheath 102 straightens the bend. After extension, the material may return to its preformed shape when the constraints of, e.g., sheath 102 have been removed. In some embodiments, elongate element 112 may be configured to transition to its second state upon exposure to some trigger, including, but not limited to, body chemistry and/or temperature.

The angle and its spacing from the distal end 206 of elongate member 112 will determine the position distal tip 206 in the second state. The degree of bend may be chosen to lie between 15-90 degrees, preferably 35-60 degrees, more preferably 45 degrees. In the first state, with elongate member 112 retracted into sheath 102, the sheath's inside walls exert a straightening force on bend portion 116 to retain the elongate member 112 in a straightened or substantially straightened configuration. Extending elongate member 112 distally, by its own action or by withdrawal of sheath 102, removes the constraining forces exerted by the sheath 102, allowing the distal portion of elongate member 112 to assume the second state.

Translating elongate member 112 into the second state allows the end-effector 114 to be positioned adjacent a target area, owing to the lateral movement of distal tip 206. A surgeon can maneuver the distal end of medical device 100 are longitudinally adjacent to a target area, but lateral movement of the distal tip is difficult. The ability to move the end-effector 114 into the second state and its considerably to the precision by which therapeutic action can be taken.

To facilitate effective positioning of the end-effector 114, the elongate member 112 may be formed of any suitable, flexible material known to those in the art. Alternatively, portion 116 may be a flexible member connected to the proximal end 104 of the elongate member 112 to connect the elongate member 112 to the end-effector 114, or the end-effector 114 itself may include a flexible portion. If the elongate member 112 is implemented as an inner tube, the tool may extend along the lumen of the inner tube and extend from the distal end of the inner tube to define the end-effector 114. By providing end-effector 114 separately from the elongate member 112, different end-effectors of varying size may be fitted to the elongate member 112 or passed along a lumen of the elongate member 112 in the form of medical tools.

Thus, flexible movement of the bend portion 116 of the elongate member 112 may be achieved by making use of a pre-formed bent distal end as discussed above. Alternatively, the bend portion 116 may move laterally by making use of a known steering mechanism. For example, a set of wires connected to proximate portion 116 may extend to the proximal end 104 of the elongate member 112 along the length of the elongate member 112 through a channel (not shown). These wires may be manipulated and the degree of lateral deflection of the proximate portion 116 may be controlled.

As discussed above, the elongate member 112 may be a hollow tube such that the end-effector 114 may be retracted into the elongate member 112 in a collapsed state and expanded once the end-effector 114 is deployed from the elongate member 112. Alternatively, the elongate member 112 may be formed as a solid tube with an end-effector 114 at its distal end 106. In such a scenario, the end-effector 114 and elongate member 112 are retracted into and deployed from the sheath 102.

The elongate member 112 may further include one or more channels (not shown) to advance additional medical devices or tools such as an ultrasound device, lithotripters, lasers, or dissection tools near the target area. Once, the requisite medical procedure has been performed, be it rupturing a desired stone or resecting a polyp, the medical rupturing or resection tool may be retracted into the elongate member 112 and a tool deployed to capture the stone or polyp fragments. Subsequently, the capturing tool may be withdrawn or the entire medical device 100 retracted to remove the dissected tissue.

The handle 110 allows operators to grip the medical device 100 for manipulation within the patient's body. The handle 110 may include one or more ports 113 for inserting tools into medical device 100. In addition to the ports 113, the handle 110 may include maneuvering means to navigate the distal end 106 of the sheath 102 within a patient's body. Such means may include mechanical levers, sliders, or pulleys; electronic buttons, switches, or joysticks, or a combination of mechanical and electronic controls. Further, the handle 110 may include control means to control the position of the elongate member 112 relative to the sheath 102 to retain the elongate member 112 at the desired configuration between the first and second states. Alternatively, the handle 110 includes actuation means for manipulating the elongate member 112 as well as the end-effector 114. For example, the end-effector 114 may be translatable between a deployed position and a retracted position. In the retraced position, end-effector 114 may lie within sheath 102 or elongate member 112; and, in the deployed position, end-effector 114 may extend distally beyond the sheath's distal end 106.

The handle 110 may be temporarily or permanently attached to the sheath's proximal end 104. Various temporary and permanent attachment means may be suitable, such as snap-fit, screw-fit, Luer-lock, gluing, welding, etc. Any such means as are currently known or may be developed in the future may be utilized without departing from the scope of the present disclosure.

As noted above, the end-effector device shown in FIGS. 1B-1D is a basket 202, which could also be presented as a cage, of similar construction. The basket 202 is shown in a compressed configuration in FIG. 1B, an expanded configuration in FIG. 1C, and in a non-expanded condition in FIG. 1D As explained below, basket 202 can be formed in a number of configurations, but all these embodiments are formed by a number of legs 204, extending from a proximal end to a distal end 208 of the basket 202.

Embodiments of the basket 202 can take the form of a zero-tip basket, as illustrated, or a conventional tipped design can be employed. The illustrated zero-tip embodiment is configured as a basket having multiple wires loops that begin from a proximal end, extend distally in loops, and then reconnect at the proximal end. Multiple wire loops cross each other at their distal ends, forming a basket with no definable distal tip. If desired for increased rigidity, the legs 204 may be joined at their distal end by permanent joining means such as, gluing, welding, etc. Other attachment means could be adopted as would be evident to those skilled in the art

In the alternative, the zero-tip basket design can be modified to provide a conventional tip. There, each leg 204 extends from proximal end to distal end 208, where the wires are joined by welding or similar attachment processes, forming a tip at distal end 208. As known in the art, a separate tip element may be attached to extend a desired distance from distal end 208. The tip element can serve simply to provide stability, or it can be used to provide standoff from a particular structure within the bodily lumen, or it could be configured as a cautery or ablation instrument, as desired. Structure and employment of such devices are well known in the art.

In either embodiment of basket 202, each leg 204 may be formed of a single wire, or alternatively, the legs 204 may be formed of multiple wires twisted or braided along the length of the basket 202. Moreover, the multi-wire legs 204 may extend along the entire length of the basket 202. In other cases, portions of the legs 204 may be formed of single wires, while other portions may be formed of multiple wires. In yet other cases, the thickness of the wires may be uniform along the lengths of the legs 204. Alternatively, the wires may be thicker in the middle and thinner at the proximal and bend portions of the legs 204, or vice-versa. In addition, projections may be present on the inward facing surfaces of legs 204. Because these projections are inward facing, they may grasp the dissected tissue when the retracting member is compressed, preventing the tissue from slipping out of the basket during retraction. In both embodiments, the number and positions of the projections may vary considerably without departing from the disclosure's scope.

In the deployed state, shown in FIG. 1C, the basket 202 may translate between a compressed configuration and an expanded configuration. In the latter configuration, the basket diameter may be sufficiently large to radially push outwardly on the tissue surrounding, e.g., a targeted object. To shift between the compressed and expanded configurations, the basket 202 may be self-expandable, or it may be expanded by any suitable means known in the art. A self-expandable basket 202 may be configured to transition to the expanded state without any assistance. That configuration is illustrated in FIG. 1C, where the basket 202 self-expands as soon as it has cleared the distal tip of sheath 102. To return the self-expandable retracting member to a compressed state, however, an external force may be required. Transition from an expanded considered configuration to a compressed configuration is generally accomplished by withdrawing elongate member 112 into sheath 102, so that the inside walls of sheath 102 to exert a compressing force against basket 202.

Clearly, sheath 202 must be constructed of material having sufficient strength to exert the required force against basket 202, or whatever end-effector 114 may be employed. If the sheath material is too thin or too elastic, it may not be sufficient to hold the self-expandable retracting member in its compressed state and the basket 202 may expand within the sheath 102 itself. Alternatively, if sheath 102 is too rigid or thick, it may not be able to traverse the circuitous path within a patient's body, causing injury. The material and thickness of the basket 202 and sheath 102 is thus chosen to keep both aspects in mind.

Expansion of basket 202 may be accomplished by the surgeon or other medical device operator, as illustrated in FIG. 1E. There, one or more pull wires 120 may be attached to the basket's distal end or proximal end, such that the operator can use appropriate controls mounted on handle 110 to pull the wires, thus exerting a force on basket 202. In that embodiment, basket 202 includes an expansion mechanism (not shown) responding to pull wires 120 to expand the basket 202. The operator can manipulate the controls to reverse that action, returning basket 202 to the compressed configuration.

Another embodiment provides alternative expansion means, such as an inflatable balloon, which can be used to expand basket 202 by exerting an outward radial force on the legs 204. Such expansion means (not shown) may include balloons inflated by fluids or dilators. Other such inflating means may also be utilized without departing from the scope of the present disclosure. For example, means such as springs or levers may be utilized to expand the member. Similarly, the member itself may be configured to include pivotal structures connected to one another. For instance, the member may be formed of multiple wires connected to one another along pivotal joints (not shown). An outward force on the pivotal joints expands the various wires connected to the joint, expanding the basket 202.

In order to avoid unwanted damage to body tissue, the expansion of basket 202 may be limited. In one embodiment, for example, the medical device 100 may include visualization aids, such as cameras or fluorescent dyes, to visualize the extent of expansion of basket 202. Further, the basket 202 may include a force or expansion-limiting component that prevents the basket 202 from expanding beyond a certain limit. Often, the expansion limit may be set during manufacture. For example, operators may know the average body cavity sizes, and may ensure that the member does not expand beyond the average lumen size.

By radially expanding, the basket 202 pushes surrounding tissue outwardly. The degree of expansion and the strength of the legs determine the cavity area created by the basket 202. For example, if the legs 204 have a very small diameter, they may not be able to push the tissue too far, as the force exerted by the legs on the tissue may be comparable to the force exerted by the tissue on the legs. On the other hand, if the diameter of the legs is relatively larger, they may be able to apply additional forces on the tissue, pushing it further away from the procedure site.

In another embodiment, the legs 204 may have a sharp cutting edge (not shown), such that the expansion member may dissect tissue during expansion. In some embodiments, the operator may rotate the sheath 102, in turn rotating the basket 202, and allowing it to behave as a scoring tool. In such an embodiment, it may be preferable to control the degree of expansion, so that the operator may control tissue dissection. According to one control technique, the operators may slowly release pull wires holding the basket 202 compressed, thereby releasing the force on the member in a controlled fashion.

FIG. 2 shows a side view of an alternative end-effector that includes a dissecting tool. Here, the end-effector 114 includes a cutting tool 302 and a basket 202, which has a proximal end and a distal end 208. The tool 302 may perform cutting operations by means of electro-cautery or simply by slicing tissue with a wire member, as shown. Electrical connections or elongated supporting members may extend through the sheath's lumen, connecting the cutting tool 302 to the proximal end of medical device 100.

In some embodiments, both the cutting tool 302 and the basket 202 may be translatable between a retracted position and a deployed position. It will be understood that the cutting tool 302 and basket 202 can switch positions independently. For example, basket 202 may be deployed while cutting tool 302 is in the retracted position, or vice versa.

The cutting tool may have a curved or bent bend portion 304, with an electrode at the distal tip. High power electrical currents are supplied to the bent bend portion 304, enabling the tool to burn or cauterize tissue for dissection. The curved bend portion 304 allows the operator to cauterize tissue at an angle to the longitudinal axis. In some instances, the bend portion 304 may be selectively pivotable relative to the remainder of the cutting 302, so that operators may configure the electrode's firing angle, as desired. Alternatively, the bend portion 304 may be straight, and may not have any provision for angular displacement.

Electrical connections may be provided to connect the proximal end of the cutting tool 302 with an internal or external power source. Operators may use power controls present on the handle 110 or on the external power supply to activate the cutting tool 302. If the power controls are present on the handle 110, the handle 110 may also include power means, such as a portable battery to activate the cutting tool 302.

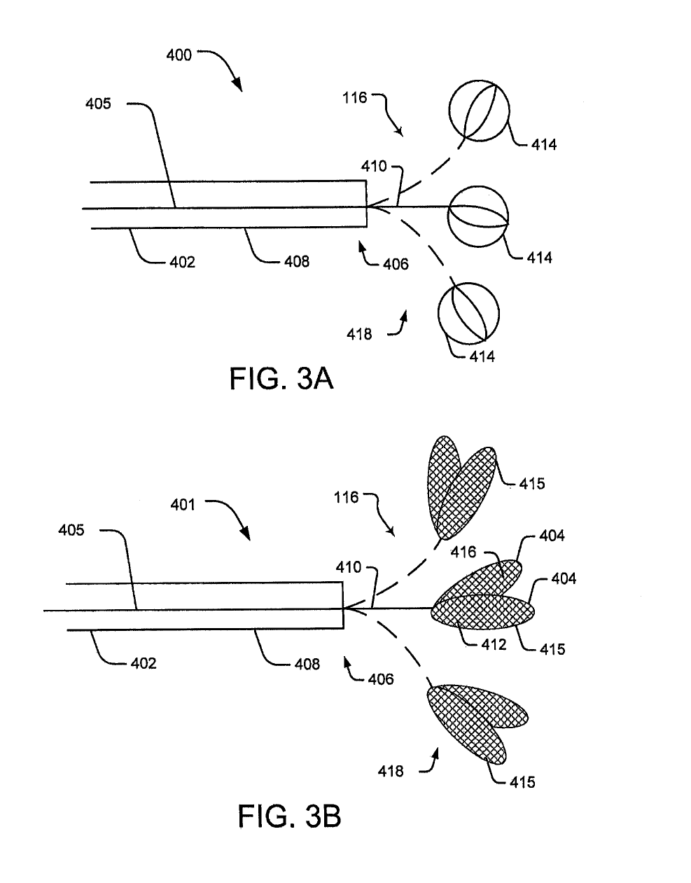

FIGS. 3A and 3B illustrate two embodiments of a fully steerable end-effector of the present disclosure. Both embodiments demonstrate the functioning of the steerable end-effector 414, e.g., produced by the bend portion 116 of elongate member 112. In some embodiments, medical devices 400 (FIG. 3A) and 401 (FIG. 3B) include an elongate member 112 with bend portion 116 formed in the vicinity of its distal end. In other embodiments, the distal portions of medical devices 400 and 401 may be articulable. The medical devices 400, 401 may further include an end-effector. In some embodiments, the end-effector may include a basket 414 or mesh grasper 415. However, any suitable end-effector may be used in accordance with the present disclosure. As noted above, extending elongate member 112 into the second state, as illustrated here, causes the distal end of the elongate member 112 to move laterally away from the longitudinal axis 405 of medical devices 400, 401. Thus, when elongate member 112 is rotated, end-effectors 414, 415 do not simply rotate in place, but in fact describe a circle around the longitudinal axis 405, as illustrated by the multiple positions of end-effectors 414, 415 shown in FIGS. 3A and 3B.

FIG. 3B depicts an alternative embodiment of an end-effector in the form of a mesh grasper 415 that comprises two expandable mesh-covered frames 404 defining a retracting member 418. The frames 404 are joined at their proximal ends. In the present embodiment, frames 404 are oval shaped, but other shapes may be provided as are deemed useful for particular therapeutic scenarios, which may call for rectangular, square, or circular graspers. In some embodiments, both frames 404 may be configured to move relative to one another. In other embodiments, one of frames 404 may be fixed while the other of frames 404 may be configured to move relative to the fixed frame.

Additional embodiments may employ any of the wide range of end-effectors known in the art. Examples of suitable end-effectors may include, but are not limited to, scissors, a snare loop, forceps, a lithotripter, or pincers. Similarly, both vision and illuminating devices may also be fitted to elongate members 112 having a bend portion 116.

A further embodiment may facilitate effective swivel movement of the end-effector 414 by providing a flexible member 410 in the vicinity of bend portion 116. Alternatively, elongate member 402 may include actuation means connected to a handle (not shown), which allows the operator to steer end-effectors 414, 415 in order to facilitate effective alignment of the end-effectors 414, 415 with the target tissue and to control the articulation of the frames 404 relative to each other. For example, a set of wires may connect end-effectors 414, 415 to proximal end of the elongate member 402 along the length of the elongate member 402. These wires may be manipulated manually or automatically, and the degree of swivel of the end-effectors 414, 415 may be selectively achieved.

The mesh portion of the end-effector 415 may be formed in a number of ways. Mesh 412 may be formed of multiple filaments 416 arranged generally in two sets of parallel lines wound in opposite directions forming a web. The filaments 416 intersect in an overlapping pattern at multiple interstices. The ends of each of the filaments 416 may be constrained to prevent the filaments from fraying or unraveling.

In other embodiments (not shown), filaments 416 may be arranged in different configurations. For example, mesh 412 may be formed by winding one set of helices in the same direction. It will be understood that many other winding patterns may be contemplated and all these patterns and configurations are within the scope of the present disclosure.

The shape, thickness, or other characteristics of filaments 416 may also vary. For example, filaments 416 may each be made from multi-filar threads woven together to form filaments having a generally round shape. Alternatively, other filament configurations may be employed, such as round wire, flat ribbon, threads, fibers, monofilament, multi-filament, or combinations thereof.

In some embodiments, a biocompatible polymer material such as silicone may coat the filaments 416, forming a complete sheath so that the dissected tissue does not poke through the gaps between the filaments.

In some embodiments, articulable jaws may be present on the frame circumference, allowing better tissue retention in the basket 202. In addition, the frames 404 may have hooks or other projections on the circumference for adhering or grasping tissue. Alternatively, cauterization features may be incorporated by addition of an electrocautery hook to the outer edges of the frames 404.

Similar to the basket 202 of FIGS. 1A-1E, the retracting member 418 translates between expanded and a compressed configurations. In the expanded configuration, its diameter may be sufficiently large to radially push outwardly on the tissue surrounding, e.g., a targeted object. Once the tissue has been dissected the planar structures move towards each other to grasp the tissue. To shift between the compressed and expanded configurations, the retracting member 418 may be expanded by any suitable means known in the art. In some embodiments, one or more pull wires 410 may be utilized. In some embodiments, a single pull wire may include a bifurcated end connected to the proximal portion of each frame. Alternatively, individual pull wires corresponding to each of the frames may be utilized.

Pull wires 410 may be attached to the retracting member's distal end or proximal end. In one embodiment, when the pull wires 410 are pulled, they exert a force on the retracting member 418, moving the distal ends of planar structures towards each other, and decreasing the cavity size. When the pull wires 410 are released, the force is released allowing the member to expand. Moreover, means to pull, push, or release the pull wires 410 may be configured in the device's handle allowing operators to easily expand or compress the member, as required. Insofar as the retracting member 418 is not self-expanding, it will be appreciated that the pull wires 410 must possess sufficient rigidity to allow the wires to be pushed in order to expand or open the jaws of the retracting member 418. Alternatively, the actuation means may be present external to handle.

A self-expandable retracting member 418 may be configured to transition to the expanded configuration without any assistance. To transition the self-expandable retracting member to a compressed or closed configuration, however, an external force may be required. Various techniques may be adopted to apply force on a self-expandable retracting member 418 and keep it in the compressed configuration. Suitable materials to form the self-expandable retracting member 418 may include shape memory alloys, such as Nitinol™, stainless steel, etc. It will be understood that any other suitable material now known or known in the future may be used to form the self-expandable member.

As depicted, the elongate member 402 may be a hollow tube such that end-effector 414 may be retracted into the elongate member 402 in a collapsed configuration and expanded out of the elongate member once the elongate member 402 is deployed. Elongate member 402 may further include one or more channels (not shown) to advance additional medical devices or tools such as ultrasound device, lithotripters, or dissection tools near the target area. Once, the requisite medical procedure ruptures a desired stone or dissects tissue, e.g., the medical tool may be retracted into the elongate member 402 and the end-effector 414 may be compressed, such that it holds the stone fragments or dissected tissue therein. Subsequently, the end-effector 414 may be withdrawn or the entire medical device 400 may be retracted proximally to remove the dissected tissue.

The end-effector, elongate tube and related components may be formed of any suitable biocompatible material currently known in the art or that may in future become available. For example, the material may be nitinol or stainless steel. The material can be selected from among those generally available to the art based on desired stiffness, resilience, and other properties of the arms.

Medical procedures, such as lithotripsy may be used to fragment a relatively large stone into smaller fragments via, e.g., acoustic vibrations or any other suitable energy source. An non-steerable end-effector in such procedures may lead to procedural delays in fragment removal and may cause additional patient discomfort. In this scenario, an end-effector 114 with flexible and steerable end-effector facilitates quicker, efficient, and collective fragment removal.

The medical devices and end-effectors described above may be employed in a method for manipulating tissue from within the body of a patient. To this end, the medical devices and end-effectors may be introduced into the body lumens of a patient through an exemplary medical device, such as an endoscopic device, a guide tube, or other catheter or introducer. Once in the endoscopic device or other introducer, the medical device 100 may be inserted into the body and directed to the desired site, at which point the distal tip of the elongate member 112 is extended from the sheath 102. As the bend portion 116 of the elongate member 112 advances from the sheath 102, the elongate member bends outward, moving the end-effector laterally outwardly from the longitudinal axis as it extends from the sheath. Next, if an end-effector includes an expandable basket, the basket may be deployed and expanded to increase the cavity size. Next, a cauterizing or lithotripter tool may be advanced through one or more working channels of the medical device 100 and activated to resect tissue or rupture stones. This is followed by the end-effector encapsulating the cauterized tissue or ruptured stone. The cauterizing or lithotripter tool may subsequently be retracted into the sheath 102 and the end-effector 114 compressed to hold the dissected tissue or ruptured stone. The medical device 100 may then be retracted proximally and removed from the endoscopic device.

It should be apparent that the medical device 100 might be used to carry out a variety of medical or non-medical procedures, including surgical and diagnostic procedures in a wide variety of bodily locations. For example, stone removal from numerous body organs, such as kidneys, and polyp removal from numerous body organs, such as the esophagus, stomach, bladder, or prostate could be accomplished using the method discussed above. In addition, at least certain aspects of the aforementioned embodiments may be combined with other aspects of the embodiments, or carried out separately without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being defined by the following claims.

1. A medical device comprising:
a tubular member having a proximal end, a distal end, and a lumen extending between the proximal end and the distal end; and
an elongate member having a proximal end and a distal end, wherein at least a portion of the elongate member is movably disposed within the lumen of the tubular member, wherein the distal end of the elongate member includes an end-effector, wherein a portion of the elongate member between the proximal end and the distal end is configured to transition from a first state to a second state when the portion is advanced out of the lumen of the tubular member, and wherein the portion is proximal of the end-effector.

2. The medical device of claim 1, wherein the portion includes a shape memory or super-elastic material.

3. The medical device of claim 1, wherein the end-effector is steerable.

4. The medical device of claim 1, wherein the portion is configured to transition from the first state to the second state without application of any additional forces.

5. The medical device of claim 1, wherein the first state is substantially straight.

6. The medical device of claim 1, wherein the second state includes a curved configuration.

7. The medical device of claim 1, wherein the portion is configured to transition from the first state to the second state upon exposure to a trigger.

8. The medical device of claim 1, wherein the trigger is one of body chemistry or temperature.

9. The medical device of claim 8, wherein the end-effector is configured to manipulate tissue or other objects within a patient's body.

10. The medical device of claim 9, wherein the end-effector includes a self-expanding basket.

11. A medical device comprising:
a tubular member having a proximal end, a distal end, and a lumen extending between the proximal end and the distal end; and
an elongate member including a shape-memory material and having a proximal end and a distal end, wherein at least a portion of the elongate member is movably disposed within the lumen of the tubular member, wherein the distal end of the elongate member includes an end-effector, wherein a portion of the elongate member between the proximal end and the distal end is configured to transition from a substantially straight configuration to a second configuration different from the substantially straight configuration when the portion is advanced out of the lumen of the tubular member, and wherein the portion is proximal of the end-effector

12. The medical device of claim 11, wherein the second configuration includes the distal end of the elongate member deflecting away from a longitudinal axis of the medical device.

13. The medical device of claim 11, wherein the portion is configured to transition from the substantially straight configuration to the second configuration upon exposure to a trigger.

14. The medical device of claim 13, wherein the trigger is one of body chemistry or temperature.

15. The medical device of claim 11, wherein the end-effector is expandable.

16. The medical device of claim 11, wherein the elongate member further includes a lumen.

17. The medical device of claim 11, wherein the end-effector is selectively steerable.

18. A method for manipulating tissue within a body of a patient, the method comprising:
introducing a medical device into the body, the medical device including:
a tubular member having a proximal end, a distal end, and a lumen extending between the proximal end and the distal end; and
an elongate member having a proximal end and a distal end, wherein at least a portion of the elongate member is movably disposed within the lumen of the tubular member, wherein the distal end of the elongate member includes an end-effector, wherein a portion of the elongate member between the proximal end and the distal end is configured to transition from a first state to a second state when the portion is advanced out of the lumen of the tubular member, and wherein the portion is proximal of the end-effector;
advancing the medical device to a desired location in the body;
deploying the elongate member by sliding the elongate member relative to the tubular member such that the portion transitions from the first state to a second state, and
manipulating the end-effector.

19. The method of claim 18, wherein the portion is substantially straight in the first state and substantially curved in the second state.

20. The method of claim 18, further comprising grasping tissue with the end-effector.
